(19) United States
(12) Patent Application Publication
(10) Pub. No.: US 2024/0424059 A1
(43) Pub. Date: Dec. 26, 2024

(54) Formulations for VEGF Receptor Fusion Proteins

(71) Applicant: LEK Pharmaceuticals d.d., Ljubljana (SI)

(72) Inventors: Aleš Žula, Ljubljana (SI); Mitja Zidar, Ljubljana (SI); Kaja Belko-Parkel, Ljubljana (SI); Roman Šink, Ljubljana (SI); Blaž Lebar, Ljubljana (SI)

(73) Assignee: Lek Pharmaceuticals d. d., Ljubljana (SI)

(21) Appl. No.: 18/834,800

(22) PCT Filed: Sep. 6, 2022
(86) PCT No.: PCT/EP2022/074711

§ 371 Date: Jul. 31, 2024

(30) Foreign Application Priority Data
Sep. 6, 2021 (EP) 21195093.6

(51) Int. Cl. A61K 38/17; A61K 47/12; A61K 47/20; A61K 47/26

(52) U.S. Cl. CPC A61K 38/179; A61K 47/12; A61K 47/20; A61K 47/26

(57) ABSTRACT

The present invention relates to a liquid formulation comprising a VEGF receptor fusion protein at a concentration of at least 100 mg/ml, and wherein the formulation comprises: i) no buffering agent, ii) a citrate buffer, or iii) a histidine buffer preferably without sucrose and preferably with methionine. In addition, the invention relates to an article of manufacture comprising a container with such liquid formulation as well as the use of the liquid formulation for a method of treatment.

The present invention relates to a liquid formulation comprising a VEGF receptor fusion protein at a concentration of at least 100 mg/ml, and wherein the formulation comprises: i) no buffering agent, ii) a citrate buffer, or iii) a histidine buffer preferably without sucrose and preferably with methionine. In addition, the invention relates to an article of manufacture comprising a container with such liquid formulation as well as the use of the liquid formulation for a method of treatment.

Fc-based fusion proteins, i.e. fusion proteins which are composed of an immunoglobin Fc domain that is linked to another peptide, are among the most relevant protein therapeutics. The fused partners have significant therapeutic potential, and they are attached to an Fc-domain to endow the hybrids with a number of additional beneficial biological and pharmacological properties. Perhaps most important, the presence of the Fc domain markedly increases their plasma half-life. Moreover, from a biophysical perspective, the Fc domain folds independently and can improve the solubility and stability of the fusion partner. Examples for Fc-based fusion proteins are vascular endothelial growth factor (VEGF) receptor Fc fusion proteins like aflibercept.

Advances in biotechnology have made it possible to produce a variety of proteins for pharmaceutical applications. However, as proteins are large and complex (i.e., possessing multiple functional groups in addition to complex three-dimensional structures), the formulation of such proteins poses special problems. Degradation pathways, such as non-native irreversible aggregation and other routes, resulting in undesired post-translational modifications, affect multiple steps in the production of these biotherapeutics, including purification, freezing, transportation and long-term storage. Not only that degraded protein molecules are often unable to perform biological activity, but also protein aggregates in particular can potentially cause serious adverse effects by enhancing pharmaceutical product's immunogenicity and thus reducing its safety and efficacy. This applies in particular for high concentration formulations. While high concentrations formulations (i.e. ≥100 mg/ml) allow for smaller injection volumes of the biotherapeutic protein, they often exhibit a tendency of increased protein aggregation and viscosity, which results in lower overall protein potency, and lower manufacturing and poorer storage stability.

Although there are some formulations for VEGF receptor Fc fusion proteins known to the skilled person, there is still an urgent need to develop new formulations, in particular for formulations with high concentrations of VEGF receptor Fc fusion proteins.

The problem to be solved by the present invention was thus to provide high concentration formulations for VEGF receptor fusion proteins like aflibercept with satisfactory stability, in particular in terms of low aggregation levels.

This problem is solved by the subject-matter as set forth below and in the appended claims.

In a first aspect, the present invention relates to a liquid pharmaceutical formulation comprising a VEGF receptor fusion protein at a concentration of at least 100 mg/ml, and wherein the formulation comprises i) no buffering agent, ii) a citrate buffer, or iii) a histidine buffer preferably without sucrose and preferably with methionine.

The formulation of the present invention is preferably a stable formulation with preferably low aggregation tendency when stored. A “stable” formulation according to the present invention is a formulation in which the VEGF receptor Fc fusion protein retains its physical stability and/or chemical stability and/or biological activity upon storage. Preferably, the VEGF receptor Fc fusion protein essentially retains its physical and chemical stability, as well as its biological activity upon storage. The storage period is generally selected based on the intended shelf-life of the formulation.

Various analytical techniques for measuring protein stability are available in the art. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact the VEGF receptor Fc fusion protein; peptide map (for example tryptic or LYS-C) analysis; evaluating biological activity or antigen binding function of the VEGF receptor Fc fusion protein; etc. Instability may involve any one or more of: aggregation, deamidation (e.g. Asn deamidation), oxidation (e.g. Met oxidation), isomerization (e.g. Asp isomerisation), clipping/hydrolysis/fragmentation (e.g. hinge region fragmentation), succinimide formation, unpaired cysteine(s), N-terminal extension, C-terminal processing, glycosylation differences, etc. According to the present invention, a stable formulation is in particular a formulation which shows after storage for 8 weeks at 25° C. an increase in aggregates by less than 1.30%, preferably less than 1.20%, most preferably less than 1.10%, as compared to before storage at 25° C. for 8 weeks.

The VEGF receptor Fc fusion protein contained in the inventive formulation can be any VEGF receptor Fc fusion protein of interest. For example, the VEGF receptor Fc fusion protein may:
be encoded by the nucleic acid sequence of SEQ ID NO: 1 or nucleotides 79-1374 or 79-1371 of SEQ ID NO: 1;
comprise amino acids of SEQ ID NO: 2 or amino acids 27-457 or 27-458 of SEQ ID NO: 2;
comprise:
(1) a VEGFR1 component comprising amino acids 27 to 129 of SEQ ID NO:2;
(2) a VEGFR2 component comprising amino acids 130 to 231 of SEQ ID NO:2;
(3) a multimerization component (“FcΔC1 (a)”) comprising amino acids 232 to 457 or 458 of SEQ ID NO:2 (the C-terminal amino acids of SEQ ID NO:2, i.e., K458, may or may not be included in the VEGF receptor fusion proteins). Note that amino acids 1 to 26 of SEQ ID No:2 are the signal sequence;
comprise an immunoglobin-like (Ig) domain 2 of a first VEGF receptor (e.g., VEGFR1) and Ig domain 3 of a second VEGF receptor (e.g., VEGFR2), optionally further including an Ig domain 4 of the second VEGF receptor (e.g., VEGFR2) and a multimerizing component (e.g., Fc domain of IgG);
be conbercept; or
be aflibercept.

In a preferred embodiment of the invention, the VEGF receptor Fc fusion protein is selected from aflibercept and conbercept. Aflibercept is a 115 kDa recombinant protein that fuses the second extracellular domain of human VEGFR-1 and the third extracellular domain of human VEGFR-2 with the Fc portion of human immunoglobulin IgG1. Conbercept is a 143 kDa recombinant anti-VEGF fusion protein engineered from a full human cDNA sequence in Chinese hamster ovary cells. The Fab of conbercept comprises the second extracellular domain of VEGFR-1 and the third and fourth extracellular domains of VEGFR-2, which then fuses to the Fc of human IgG1. Most preferably, the VEGF receptor Fc fusion protein in the inventive formulation is aflibercept.

The VEGF receptor Fc fusion protein, in particular aflibercept, is present in the inventive formulation in a concentration of at least 100 mg/ml (high concentration formulation). The VEGF receptor Fc fusion protein, in particular aflibercept, may be present in a concentration of at least about 105 mg/ml, at least about 110 mg/ml, at least about 115 mg/ml, at least about 120, mg/ml, at least about 130 mg/ml, at least about 140 mg/ml, at least about 150 mg/ml, at least about 160 mg/ml, at least about 170 mg/ml, at least about 180 mg/ml, at least about 190 mg/ml, at least about 200 mg/ml, at least about 250 mg/ml, or at least about 300 mg/ml. The VEGF receptor Fc fusion protein, in particular aflibercept, may for example be present in a concentration range from about 100 mg/ml to about 200 mg/ml, from about 105 mg/ml to about 150 mg/ml, from about 100 mg/ml to about 120 mg/ml, or from about 110 mg/ml to about 120 mg/ml. Most preferably, in particular in case where the VEGF receptor Fc fusion protein is aflibercept, the fusion protein is present in a concentration of about 115 mg/ml. The fusion protein, in particular in the case of aflibercept, may for example have a concentration of 114 to 116 mg/ml, in particular 114 to 115 mg/ml. In some embodiments where the fusion protein is aflibercept the concentration thereof may be about 114.3 mg/ml.

The inventive formulation comprises i) no buffering agent, ii) a citrate buffer, or iii) a histidine buffer with optionally methionine and preferably without sucrose. In embodiments where the formulation comprises no buffering agent, substantially all buffering capacity is provided by the VEGF receptor Fc fusion protein itself and other typical buffers such as histidine, acetate, citrate, TRIS, or phosphate buffers are not present, or at least only preset in concentrations which are not sufficient to sufficiently buffer the formulations. Such formulation without further buffering agent is typically termed in the art a “self-buffered” formulation (Gokarn, Yatin R., et al. “Self-buffering antibody formulations.” Journal of pharmaceutical sciences 97.8 (2008): 3051-3066). The inventors have demonstrated that such self-buffered formulations provide for good storage stability for VEGF receptor Fc fusion proteins as regards aggregation formation. Preferably, such self-buffered formulation has a pH between about pH 5.0 to 6.5, about pH 5.5 to 6.2, or about pH 5.8. In the alternative, the inventive formulation may comprise a citrate buffer, which also provides for low aggregation. A “citrate buffer” is a buffer comprising citrate ions. Examples of citrate buffers include sodium citrate, ammonium citrate, calcium citrate, magnesium citrate and potassium citrate solutions. The citrate buffer has preferably a pH between about pH 5.0 to 6.5, about pH 5.5 to 6.2, or about pH 5.8. Preferably, the concentration of a citrate buffer is in the range from about 5 to about 20 mM, more preferably about 10 to about 20 mM. Finally, the formulation may comprise in a third alternative a histidine buffer, but will in particular in this case preferably not comprise sucrose. A “histidine buffer” is a buffer comprising histidine ions. Examples of histidine buffers include histidine chloride, histidine acetate, histidine phosphate, and histidine sulfate solutions, with histidine chloride (His/HCl) being particularly preferred. The histidine buffer or histidine-HCl buffer has preferably a pH between about pH 5.0 to 6.5, about pH 5.5 to 6.2, or about pH 5.8. Preferably, the concentration of a histidine buffer is in the range from about 5 to about 20 mM, more preferably in the range of about 10 to about 20 mM. It is understood that in the context of the present invention formulations without buffering agent are in particular those, which do not comprise histidine, acetate, citrate, TRIS, and/or phosphate as buffering agent, in particular do not comprise citrate and histidine as buffering agent, or at least none of these buffers in concentrations contributing significantly to the buffer capacity of the formulation.

pH is measured using standard glass bulb pH meter. Unless otherwise indicated, the pH values and ranges defined herein for formulations of the present invention reflect the pH at 25° C. In particular in cases where the VEGF receptor Fc fusion protein is aflibercept, the pH of the formulation is preferably pH 5.8.

Aside of the buffer (or its absence in self-buffered formulations), the inventive formulation may comprise further components. For instance, the formulation may comprise one or more free amino acids (i.e. as individual compounds, not as component of a peptide or protein). However, histidine is, unless a histidine buffer is used, preferably not among these amino acids. The formulation may comprise for example one or more amino acids selected from a first group of amino acids consisting of glycine, alanine, valine, leucine, isoleucine, methionine, proline, phenylalanine and tryptophan. Preferably, the one or more amino acids are selected from proline and methionine. The concentrations of the one or more amino acids may, independently of each other, be in the range of about 5 to about 450 mM. For example, the concentration of one or more of the amino acids may be about 10 mM, about 20 mM, about 25 mM, about 35 mM, about 40 mM, about 50 mM, about 75 mM, about 100 mM, about 125 mM, about 150 mM, about 200 mM, about 250 mM, about 300 mM, about 350 mM, about 400 mM or about 450 mM. Methionine is particularly useful as antioxidant. Preferred concentrations of methionine are in the range of 5 to 25 mM. Most preferably, methionine is used in a concentration of 10 mM. If proline is used, and no sugar (or sugar alcohol) is used, then the concentration of proline may be in the range of 100 to 450 mM, in particular in the range of 250 to 300 mM. In the alternative, or in addition to one or more amino acids selected from the first group of amino acids, the inventive formulation may comprise one or more amino acids selected from a second group of amino acids consisting of arginine and lysine. Preferably, the amino acid from the second group is arginine. Arginine is particularly useful as viscosity reducing agent. Preferred concentrations of arginine are in the range of 25 to 75 mM. Most preferably, arginine is used in a concentration of about 50 mM. Lysine is also suitable as viscosity reducing agent, with preferred concentrations from about 50 to about 150 mM, with 100 mM being most preferred. Preferred combinations of free amino acids are i) methionine plus arginine, ii) proline plus methionine, iii) proline plus arginine, iv) methionine plus arginine plus proline, v) methionine plus lysine, and vi) methionine plus lysine plus proline.

Preferred combinations of buffer and amino acid are a histidine buffer and methionine or a citrate buffer and methionine.

As a further possible but optional component the formulation may comprise a surfactant. The surfactant can be in principle any (non-ionic) surfactant suitable for protein formulation, but it is preferably, selected from the group of polysorbate 20, polysorbate 80, a poloxamer (such as poloxamer 188) and combinations thereof. Most preferably, the surfactant is polysorbate 20 or polysorbate 80. Typically, but not necessarily, the surfactant, and in particular polysorbate 20 or polysorbate 80, will be present in a concentration of about 0.01% to about 0.1% w/v. Formulations of the present invention may for example comprise about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml or about 1 mg/ml. In preferred embodiments of the invention, the surfactant is chosen from polysorbate 20 and polysorbate 80, and is present in a concentration of about 0.1 mg/ml to about 0.3 mg/ml. More preferably, the concentration of polysorbate 20 and polysorbate 80 is about 0.2 mg/ml.

A further but optional component of the inventive formulation is a stabilizer selected from the group of sugars and sugar alcohols. Examples of sugars are lactose, fructose, maltose, trehalose and sucrose. The sugar may be sucrose (except in cases where the formulation is a histidine buffer: there the stabilizer is preferably not sucrose). Preferred examples of sugar alcohols are arabitol, mannitol and sorbitol. Sorbitol can help to suppress aggregation. In some embodiments, sorbitol is present in a concentration no greater than approximately 300 mM. The concentration of sorbitol can for example be in the range of 100 to 350 mM, more preferably in the range of 100 to 300 mM. If the inventive formulation contains sucrose, it is preferably used in the range of 100 to 300 mM sucrose, preferably in the range of 140 to 210 mM sucrose, preferably 146 mM sucrose. However, the present invention also specifically contemplates to use formulations according to the present invention without sucrose. Typical embodiments for such scenario are formulations which do neither comprise a buffering agent nor sucrose.

A further, optional, component of the inventive formulation is a chelator and/or oxygen-scavenger or combinations of both. Typical chelators are citrate, diethylenetriaminepentaacetic acid (DTPA), and ethylenediaminetetraacetic acid (EDTA). Examples for oxygen scavengers are methionine, sodium thiosulfate, catalase, or platinum, wherein methionine is the preferred oxygen scavenger for the present invention. In case the inventive formulation comprises a chelator, such as DTPA or EDTA, the such chelator may be present in a concentration in the range of 5 to 150 μM, preferably in the range of 20 to 100 μM. Particularly preferred concentrations are 20 μM, 50 μM, and 100 μM. Most preferred is a chelator concentration of 20 μM. In case the inventive formulation comprises an oxygen scavenger such as methionine, then such scavenger is preferably present in a concentration of about 100 μM to 15 mM. A particularly preferred concentration of methionine is 10 mM. Instead of using a chelator/and or oxygen scavenger, one may also consider to flush the headspace of the container containing the inventive formulation with an inert gas, e.g. with nitrogen, in order to displace oxygen and to minimize oxidation.

Finally, the inventive formulation may optionally also comprise an inorganic salt such as sodium sulphate or sodium chloride or combinations thereof. Sodium chloride can reduce the viscosity of a formulation. The inventive formulation may—without being limited thereto-comprise 10 to 100 mM salt, for example 10 to 20 mM sodium sulphate, in particular 16 mM sodium sulphate, or may comprise 25 to 75 mM sodium chloride, in particular 40 to 60 mM sodium chloride, in particular 50 mM sodium chloride. However, using a salt is optional and the present invention contemplates formulations without salt. e.g. without sodium chloride.

Formulations of the present invention may for example be selected from the following formulations comprising:
i) 100 to 120 mg aflibercept, in particular 114 to 115 mg aflibercept, no buffering agent, pH 5.8, proline and, optionally, methionine and/or a surfactant; or
ii) 100 to 120 mg aflibercept, in particular 114 to 115 mg aflibercept, a citrate buffer, pH 5.8, sucrose, arginine and, optionally, methionine and/or a surfactant.

Further specific examples for formulations of the present invention may for instance be selected from the following group:
i) 100 to 120 mg aflibercept, preferably 114 mg aflibercept, a citrate buffer, pH 5.8, sucrose or trehalose, polysorbate 20 (e.g. 0.3 mg/ml), and arginine and preferably no histidine;
ii) 100 to 120 mg aflibercept, preferably 114 mg aflibercept, no buffering agent, pH 5.8, proline, arginine, polysorbate 20 (e.g. 0.3 mg/ml), said formulation preferably not comprising any sugar or sugar alcohol, and
iii) 100 to 120 mg aflibercept, preferably 114 mg aflibercept, no buffering agent, pH 5.8, proline, methionine, polysorbate 20 (e.g. 0.3 mg/ml), said formulation preferably not comprising any sugar or sugar alcohol.

In a further aspect, the present invention relates to articles of manufacture comprising containers and injection devices (which may be sterile) containing a liquid formulation according to the present invention and optionally instructions for its use. The article of manufacture may in particular be a container like a glass vial or test tube, an injection device such as a pre-filled syringe (e.g., a filled glass syringe or filled plastic syringe), or an intravitreal implant, e.g., a refillable implant.

A “pre-filled” syringe is a syringe which is filled with a formulation of the present invention prior to sale or use by the physician or patient. It could contain one or more dose line graduations and/or is a dose metering system.

“Sterile” herein refers to aseptic or free from substantially all, or all, living microorganisms and their spores.

Containers and injection devices may be coated with silicone (e.g., silicone oil or baked-silicone (e.g., <40 pg or <100 pg), are substantially metal-free or substantially tungsten-free or low-tungsten.

Finally, the present invention relates to a formulation according to the present invention for use in a method of treating a disease of the human or animal body, in particular wherein the disease is a VEGF related disease such as angiongenic eye disorders or cancer.

Angiogenic eye disorders herein refer to any disease of the eye which is caused by or associated with the growth or proliferation of blood vessels and/or by blood vessel leakage. Non-limiting examples of angiogenic eye disorders that are treatable or preventable using the formulations and methods herein, include but: neovascular (wet) age-related macular degeneration (AMD), macular edema, macular edema following retinal vein occlusion, retinal vein occlusion (RVO), diabetic macular edema (DME) choroidal neovascularization (CNV), iris neovascularization, neovascular glaucoma, post-surgical fibrosis in glaucoma, proliferative vitreoretinopathy (PVR), optic disc neovascularization, comeal neovascularization, retinal neovascularization, vitreal neovascularization, vascular retinopathy, diabetic retinopathies (e.g. non-proliferative diabetic retinopathy or proliferative diabetic retinopathy, e.g., in a subject that does not suffer from DME) and or diabetic retinopathy in a patient who has diabetic macular edema (DME).

The cancer may be any type of cancer for which VEGF receptor Fc fusion proteins are therapeutically effective. Cancers include those whose growth, proliferation, survival and/or metastasis is dependent, to a degree, on angiogenesis. In an embodiment of the invention, the cancer is colorectal cancer, lung cancer, skin cancer, breast cancer, brain cancer, stomach cancer, renal cancer, prostate cancer, liver cancer or pancreatic cancer.

The liquid composition may be administered through an oral or parenteral route. In case of parenteral administration (for example, injection), it may be administered by intravenous administration, subcutaneous administration, intramuscular administration, intraperitoneal administration, endothelial administration, local administration, intranasal administration, intrapulmonary administration, intrectal administration, intratumoral administration, intravitreal administration, etc.

In a specific embodiment, the liquid composition may be an ophthalmic solution comprising a VEGF antagonist as described above, and in this case, may be parenteral formulation to be administered into a vitreous humor of an eye.

The term “comprising”, as used herein, shall not be construed as being limited to the meaning “consisting of” (i.e. excluding the presence of additional other matter). Rather, “comprising” implies that optionally additional matter, features or steps may be present. The term “comprising” encompasses as particularly envisioned embodiments falling within its scope “consisting of” (i.e. excluding the presence of additional other matter) and “comprising but not consisting of” (i.e. requiring the presence of additional other matter, features or steps), with the former being more preferred.

The use of the word “a” or “an”, when used herein, may mean “one,” but it is also consistent with the meaning of “one or more,” “at least one,” and “one or more than one.”

### FIGURES

In the following a brief description of the appended figures will be given. The figures are intended to illustrate the present invention in more detail. However, it is not intended to limit the scope of the invention to only these specifically illustrated embodiments.

FIG. 1: provides a table illustrating various formulations of aflibercept (each with 115 mg/ml) and their impact on aggregate formation after storage for eight weeks at 25° C.

FIG. 2: illustrates score contributions of different buffers/excipients for aflibercept. Score of each buffer/excipient represents its effect on reducing aggregation (in % of aggregates). The lower the score contribution, the better the effect on protein stability.

### EXAMPLES

In the following, a specific example illustrating various embodiments and aspects of the invention is presented. However, the present invention shall not to be limited in scope by the specific embodiment described herein. Indeed, various modifications of the invention in addition to those described herein will become readily apparent to those skilled in the art from the foregoing description, accompanying figures and the example below. All such modifications fall within the scope of the appended claims.

### Example 1: Aggregate Formation in Various Aflibercept Formulations

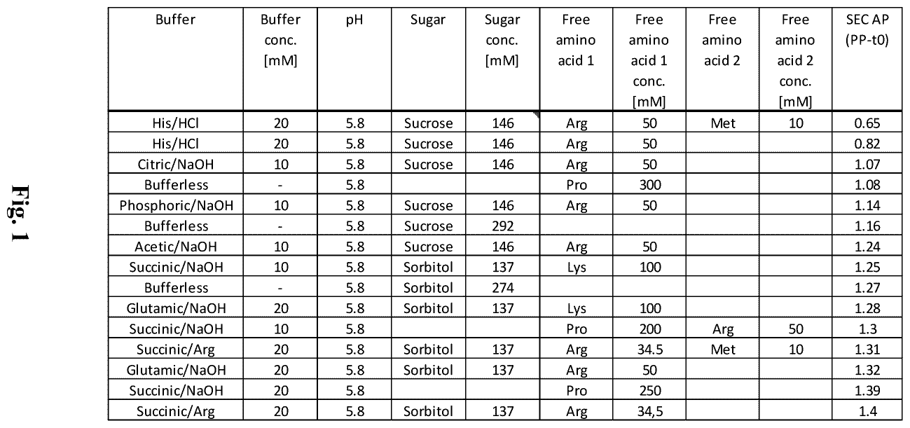

The objective of this study was to evaluate the effects of different buffers and excipients (sugars, antioxidants, viscosity reducing agents (VRAs) on aflibercept stability. Briefly, preparation of samples included pH adjustment, concentration of protein material above target concentration (using 50K Amicon® Ultra centrifugal filter tubes (Merck Millipore) and centrifuging until concentrations of 150 mg/mL are reached), and dilution with excipient stocks to target concentration (115 mg/ml), see FIG. 1. Furthermore, samples were exposed to temperature stress conditions for eight weeks at 25° C. Samples taken before (to) and after eight weeks at 25° C. were used for size exclusion chromatography (SEC) analysis and assessment of aggregation and degradation products.

For SEC analysis, samples were analysed at 40° C. on a Waters™ ACQUITY UPLC System with a SEC column (200 Å pore size, 1.7 mm bead size and 4.6 mm×150 mm column dimensions). Sample load was 0.75 μl. The mobile phase (50 mM sodium dihydrogen phosphate and 400 mM sodium perchlorate, pH 6.0) flow rate was 0.4 mL/min with a total run time of 5 min. Samples were diluted to 1 mg/ml in 150 mM sodium phosphate, pH 7, and held at 2-8° C. in the autosampler prior to injection. The data was analysed with Empower 3 software. The variability of the relative aggregate content measurement (aggregates/monomer) at described column loading was estimated to be 0.1% (absolute value).

The results, provided as increase of aggregate products (in %) of temperature stressed samples after 8 weeks (pull point; PP) at 25° C. as compared to aggregates present at to (before temperature stress exposure), are given in the table in FIG. 1.

In addition, the score contribution of the individual formulation components (buffers/excipients, see table in FIG. 1) on reducing aggregation has been evaluated. Briefly, the results were evaluated with linear regression, elucidating excipient contributions. It must be noted that this approach assumes that contributions are linearly additive. Formulation components were used as features for the fit (“one-hot encoding”). Deviation from the average aggregate increase (SEC AP column in table in FIG. 1) was used as response to which the linear function was fitted. Fit coefficients of individual formulation components are defined as their scores—the lower the value, the better the excipient. Bufferless formulation was treated as having no buffer component (“zero contribution”). A positive score indicates an increase in aggregate formation as compared to bufferless formulation while a negative score implies less aggregate formation than in bufferless formulations.

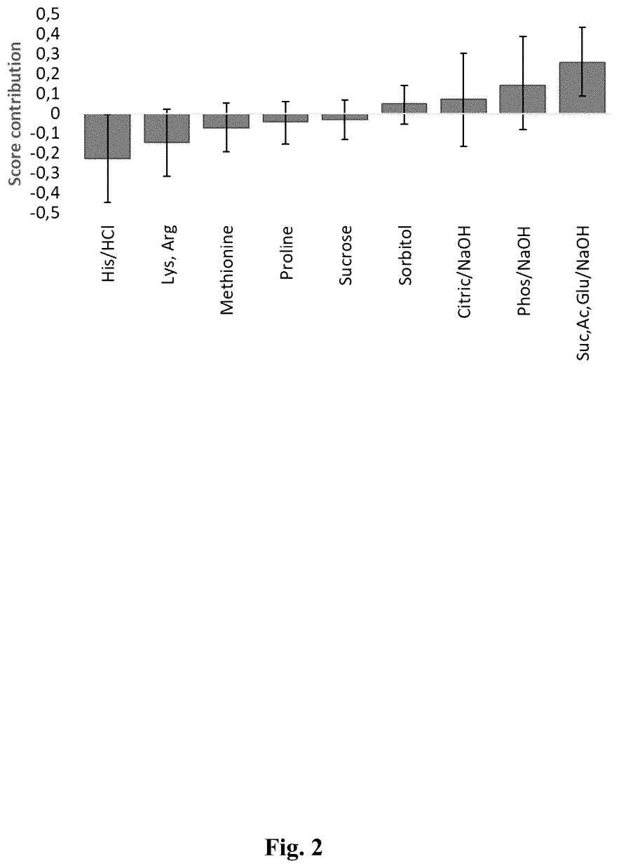

The individual contribution of the various formulation components to aggregation are depicted in FIG. 2. As can be taken from said figure, histidine buffer, bufferless and, to a lower extent, citric acid buffers are preferred buffer choices for high concentration aflibercept formulations. Moreover, addition of free amino acids such as lysine, arginine, methionine or proline is also advantageous in general.

1. A liquid pharmaceutical formulation comprising a VEGF receptor fusion protein at a concentration of at least 100 mg/ml, and wherein the formulation comprises:
i) no buffering agent,
ii) a citrate buffer, or
iii) a histidine buffer with methionine.

2. The liquid formulation of claim 1, wherein the formulation does not comprise a buffering agent.

3. The liquid formulation of claim 1, wherein the formulation comprises no buffering agent or comprises a citrate buffer, and wherein in addition the formulation does not comprise histidine.

4. The liquid formulation of claim 1, wherein the formulation comprises one or more free amino acids.

5. The liquid formulation of claim 1, wherein the VEGF receptor fusion protein is aflibercept.

6. The liquid formulation of claim 1, wherein the concentration of the VEGF receptor protein is about 115 mg/ml.

7. The liquid formulation of claim 5, wherein the concentration of aflibercept is about 115 mg/ml.

8. The liquid formulation of claim 5, wherein the concentration of aflibercept is 114 to 115 mg/ml.

9. The liquid formulation of claim 5, wherein the concentration of aflibercept is 114.3 mg/ml.

10. The liquid formulation of claim 1, wherein the formulation comprises one or more amino acids selected from the group consisting of glycine, alanine, valine, leucine, isoleucine, methionine, proline, phenylamine and tryptophan.

11. The liquid formulation of claim 1, wherein the formulation comprises methionine.

12. The liquid formulation of claim 1, wherein the formulation does not comprise sucrose.

13. The liquid formulation of claim 1, wherein the formulation comprises proline.

14. The liquid formulation of claim 1, wherein the formulation comprises one or more amino acids selected from the group consisting of lysine and arginine.

15. The liquid formulation of claim 1, wherein the formulation comprises a surfactant.

16. The liquid formulation of claim 1, wherein the formulation comprises polysorbate 80.

17. The liquid formulation of claim 1, wherein the formulation comprises polysorbate 20.

18. The liquid formulation of claim 1, wherein the formulation shows after storage for 8 weeks at 25° C. an increase in aggregates by less than 1.30%.

19. The liquid formulation of claim 1, wherein the pH of the formulation is 5.8.

20. The liquid formulation of claim 1, wherein the histidine buffer or citrate buffer has a concentration of 5 to 20 mM.

21. The liquid formulation of claim 1, wherein the formulation comprises aflibercept, a citrate buffer, pH 5.8, sucrose or trehalose, polysorbate 20, arginine and no histidine.

22. The liquid formulation of claim 1, wherein the formulation comprises aflibercept, no buffering agent, pH 5.8, proline, arginine, and polysorbate 20.

23. The liquid formulation of claim 1, wherein the formulation comprises aflibercept, no buffering agent, pH 5.8, proline, methionine, and polysorbate 20.

24. An article of manufacture comprising a container with a liquid pharmaceutical formulation of claim 1, and instructions for its use.

25. The article of claim 24, wherein the article is a pre-filled syringe or glass vial.

26. The liquid pharmaceutical formulation of claim 1, wherein the VEGF receptor fusion protein for use in a method of treating a disease of the human or animal body.


SEQUENCE LISTING (2 sequences)

SEQ ID NO: 1 - DNA, 1377 bp, Homo sapiens
  source 1..1377: mol_type = other DNA
atggtcagctactgggacaccggggtcctgctgtgcgcgctgctcagctgtctgcttctcacaggatctagttccggaagtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 458 aa, Homo sapiens
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK